(19) United States
(12) Patent Application Publication
(10) Pub. No.: US 2005/0215882 A1
(43) Pub. Date: Sep. 29, 2005

(54) Noninvasive method to determine fat content of tissues using MRI

(75) Inventors: Thomas L. Chenevert, Ann Arbor, MI (US); Hero K. Hussain, Ann Arbor, MI (US); Scott D. Swanson, Ann Arbor, MI (US); Vikas Gulani, Ann Arbor, MI (US)

Correspondence Address: MEDLEN & CARROLL, LLP, Suite 350, 101 Howard Street, San Francisco, CA, 94105, US

(73) Assignee: The Regents of the University of Michigan, Ann Arbor, MI (US)

(21) Appl. No.: 10/807,531

(22) Filed: Mar. 23, 2004

(51) Int. Cl. A61B 5/05; G01V 3/00

(52) U.S. Cl. 600/410

(57) ABSTRACT

The present invention relates to systems and methods for generating high resolution MR images of the fractional amount or percentage of fat content in an imaged object. In particular, the present invention relates to systems and methods for obtaining high resolution MR images of fat content with reduced NMR relaxation effects. Moreover, the present invention relates to systems and methods for obtaining high resolution MR images of fat content with reduced fat-percentage ambiguity. Furthermore, the present invention relates to systems and methods for diagnostic, and prognostic imaging where knowledge of fat content within tissues, organs, and lesions is beneficial to improve diagnostic accuracy, aid in risk assessment of future disease, and aid in assessment of therapeutic intervention.

This invention was supported in part with NIH grant CA 85878. The United States government may have rights in this invention.

### FIELD OF THE INVENTION

The present invention relates to systems and methods for generating high resolution MR images of the fractional amount or percentage of fat content in an imaged object. In particular, the present invention relates to systems and methods for obtaining high resolution MR images with reduced NMR relaxation effects. Moreover, the present invention relates to systems and methods for obtaining high resolution MR images with reduced fat-percentage ambiguity. Furthermore, the present invention relates to systems and methods for diagnostic and prognostic imaging where knowledge of fat content within tissues, organs, and lesions is beneficial to improve diagnostic accuracy, aid in risk assessment of future disease, and aid in assessment of therapeutic intervention.

### BACKGROUND

Magnetic resonance imaging (MRI) is an imaging technique that provides tomographic pictures of a sample (e.g., organs, tissues and structures inside the body). It does this by using a magnetic field and pulses of radio wave energy. In many cases, MRI provides information that cannot be obtained from X-ray tests. For an MRI exam, the area of the body being studied is positioned inside a strong magnetic field. MRI is an extremely flexible modality capable of generating high quality images of normal patient anatomy as well as alterations in tissues/organs due to abnormality and disease. The two main constituents that contribute to the image on clinical MRI systems are tissue water and fat. This makes MRI most useful for detecting conditions that alter the normal balance and distribution of water and fat in tissues, such as inflammation, infection, tumors, fibrosis, fatty infiltration, and injury. Information from an MRI scan can be saved and stored on a computer for further study. Photographs or films of selected views can also be made.

Despite the fact that the main constituents of MRI signals are tissue water and fat, the inability to accurately determine the relative composition of water and fat is a major limitation within the current state of imaging technology. Current clinical MR imaging techniques do not attempt to yield images of water and fat content. The most widely-used clinical MRI technique that relates to water and fat is referred to as “in-phase and out-phase” imaging. Usually a visual comparison of in-phase and out-phase images allows the interpreter to detect when there is a mixture of fat and water in tissue. In addition, in-phase and out-phase images may be added and subtracted to approximate “water-only” and “fat-only” images, but the amount and proportion of water and fat are not accurately represented in these images. There are three reasons for this limitation within the art. First, methods aimed at combining the magnitude of in-phase and out-phase signals provide images wherein it is impossible to determine which signal (water or fat) is dominant. Second, generation of fat-only images and water-only images fail to provide quantitatively accurate images of water and fat in terms of percentages. Third, T1, T2, and T2* NMR relaxation effects confound estimations of fat content percentage with a tissue.

What are needed are systems and methods of obtaining high resolution MR images with reduced NMR relaxation effects. In addition, systems and methods are needed for obtaining MR images with reduced fat-percentage ambiguity.

### SUMMARY

The present invention relates to systems and methods for generating high resolution MR images that accurately depict the fractional amount or percentage of fat content in organs, tissues, and lesions. In particular, the present invention relates to systems and methods for obtaining high resolution MR images of fat content with reduced NMR relaxation effects. Moreover, the present invention relates to systems and methods for obtaining high resolution MR images of fat content with reduced fat-percentage ambiguity. Furthermore, the present invention relates to systems and methods for diagnostic, prognostic, and therapy response imaging.

In certain embodiments, the present invention is used for diagnostic purposes. In preferred embodiments, interpretation of fat content is useful to determine the presence or absence of a specific disease. In other preferred embodiments, interpretation of fat content is useful to limit a diagnosis to several diseases. In other preferred embodiments, interpretation of fat content is used to “grade” the severity of a disease.

In certain embodiments, the present invention is used for prognostic purposes. In certain embodiments, interpretation of fat content is used to assess a risk of developing a future disease. In other preferred embodiments, interpretation of fat content is used to assess the overall health of a subject. In other preferred embodiments, interpretation of fat content is used to predict the lifespan for a subject. In other preferred embodiments, interpretation of fat content is used as a “risk factor.” In other preferred embodiments, interpretation of fat content is used in measuring overall “breast density.” In further embodiments, interpretation of fat content is used in predicting a subject's odds of developing breast cancer.

In certain embodiments, the present invention is used in assessing the effectiveness of an intervention. In preferred embodiments, the present invention is used in assessing the effectiveness of a therapeutic intervention. In other preferred embodiments, interpretation of fat content is used in assessing the degree of treatment success or “therapy response.”

In certain preferred embodiments, the present invention provides a system, comprising software. In further embodiments, the system further comprises an MRI device. In some embodiments, the software is configured to receive data obtained from the MRI device, wherein the data comprise at least one pair of consecutive in-phase and out-phase echos of a sample, wherein the software is further configured to process the at least one pair of consecutive in-phase and out-phase echos, wherein the processing comprises generating a percent of fat content within a sample, wherein the software is further configured to display the fat percentage within the sample. In some embodiments, the sample is a human head and neck, chest, abdomen, pelvis, or extremity. In other embodiments, the sample is an organ within the body, such as the liver. In other embodiments, the sample is normal tissue, or abnormal tissue, such as a lesion within the body.

In further embodiments, the data obtained from said MRI device comprise at least one pair of consecutive in-phase and out-phase images obtained with a low flip angle and at least one pair of consecutive in-phase and out-phase images obtained with a high flip angle. In further embodiments, the low flip angle setting is approximately 20 degrees, and the high flip angle is approximately 70 degrees.

In further embodiments, the MRI device is configured to analyze data from a clinical pulse sequence, wherein this data comprises a corrected T2* NMR relaxation effect value, wherein the corrected T2* NMR relaxation effect value is obtained through processing consecutive in-phase echos (Sin-phase1 and Sin-phase2) or consecutive out-phase echos (Sout-phase1 and Sout-phase2) of said sample as follows. When the first in-phase image (Sin-phase1) is recorded at a later echo time relative to the first out-phase image (Sout-phase1), the in-phase image is corrected for T2* relaxation effects by application of the following equations:

$$Sin - {phase\_T2}^{*} corrected = Sin - phase1 \cdot \sqrt{Sin - phase1 / Sin - phase2} . ; or$$

$$Sin - {phase\_T2}^{*} corrected = Sin - phase1 \cdot \sqrt{Sout - phase1 / Sout - phase2} .$$

Alternatively, when the first out-phase image (Sout-phase1) is recorded at a later echo time relative to the first in-phase image (Sin-phase1), the out-phase image is corrected for T2* relaxation effects by application of the following equations:

$$Sout - {phase\_T2}^{*} corrected = Sout - phase1 \cdot \sqrt{Sin - phase1 / Sin - phase2} . ; or$$

$$Sout - {phase\_T2}^{*} corrected = Sout - phase1 \cdot \sqrt{Sout - phase1 / Sout - phase2} .$$

In certain preferred embodiments, the present invention provides an MRI device, and software, wherein the software is configured to receive images obtained from the MRI device, wherein the images comprise at least one pair of consecutive in-phase and out-phase echos of a sample, wherein the software is further configured to process at least one pair of consecutive in-phase and out-phase echos, wherein the process comprises generating the percent of fat content within a sample, wherein the software is further configured to display the images, wherein the display presents said fat content within the sample. In further embodiments, the sample is a human abdomen. In other embodiments, the sample is a human head and neck, chest, abdomen, pelvis, or extremity. In other embodiments, the sample is an organ within the body, such as the liver. In other embodiments, the sample is normal tissue, abnormal tissue, or lesion within the body.

In further embodiments, the images obtained from the MRI device comprise at least one pair of consecutive in-phase and out-phase images obtained with a low flip angle and at least one pair of consecutive in-phase and out-phase images obtained with a high flip angle. In other embodiments, the low flip angle setting is approximately 20 degrees. In other embodiments, the high flip angle is approximately 70 degrees.

In some embodiments, the MRI device comprises a clinical pulse sequence, wherein the clinical pulse sequence comprises a corrected T2* NMR relaxation effect value, wherein the corrected T2* NMR relaxation effect value is obtained through processing consecutive in-phase sample signals or consecutive out-phase signals of the sample. In further embodiments, the processing consecutive in-phase sample signals or consecutive out-phase signals of said sample comprises application of the following equations:

$$Sin - {phase\_T2}^{*} corrected = Sin - phase1 \cdot \sqrt{Sin - phase1 / Sin - phase2} ; or$$

$$Sin - {phase\_T2}^{*} corrected = Sin - phase1 \cdot \sqrt{Sout - phase1 / Sout - phase2} ; or$$

$$Sout - {phase\_T2}^{*} corrected = Sout - phase1 \cdot \sqrt{Sin - phase1 / Sin - phase2} ; or$$

$$Sout - {phase\_T2}^{*} corrected = Sout - phase1 \cdot \sqrt{Sout - phase1 / Sout - phase2} .$$

In certain embodiment, the present invention provides a system providing a sample and an MRI device. In some embodiments, the MRI device comprises a clinical pulse sequence, wherein the clinical pulse sequence comprises a corrected T2* NMR relaxation effect value, wherein the corrected T2* NMR relaxation effect value is obtained through processing consecutive in-phase sample signals or consecutive out-phase signals of the sample. In further embodiments, the sample is a human abdomen. In even further embodiments, the sample is a human liver.

In certain embodiments, the present invention provides a method of imaging, comprising providing a sample and an MRI device. In preferred embodiments, the imaging of the sample with the MRI device comprises obtaining at least one pair of consecutive in-phase and out-phase echos (e.g., sample signals) of the sample. In further embodiments, the processing of the at least one pair of consecutive in-phase and out-phase echos of the sample generates percent of fat content data within the sample. In even further embodiments, the processed at least one pair of consecutive in-phase and out-phase echos of the sample are displayed. In even further embodiments, the displaying comprises providing an image showing the percent of fat content within the sample. In further embodiments, the sample is a human head and neck, chest, abdomen, pelvis, or extremity. In other embodiments, the sample is an organ within the body, such as the liver. In other embodiments, the sample is normal tissue, abnormal tissue, or lesion within the body.

In further preferred embodiments, the at least one pair of consecutive in-phase and out-phase echos of the sample comprises at least one pair of consecutive in-phase and out-phase images obtained with a low flip angle and at least one pair of consecutive in-phase and out-phase images obtained with a high flip angle. In some embodiments, the low flip angle setting is approximately 20 degrees. In other embodiments, the high flip angle setting is approximately 70 degrees.

In further embodiments, the processing comprises detecting the apparent fat-percentage of the at least one pair of consecutive in-phase and out-phase images of the sample obtained with a low flip angle setting and at least one pair of consecutive in-phase and out-phase images of the sample obtained with a high flip angle setting. In further embodiments, the processing comprises identifying the dominant proton species within the sample, wherein the water protons are dominant when the apparent fat-percentage increases as flip angle is increased for a sample image, wherein the fat protons are dominant when the fat-percentage decreases as flip angle is increased for a sample image.

In even further embodiments, the MRI imaging device comprises a clinical pulse sequence, wherein the clinical pulse sequence comprises a corrected T2* NMR relaxation effect value, wherein the corrected T2* NMR relaxation effect value is obtained through processing consecutive in-phase sample signals or consecutive out-phase signals of the sample. In further embodiments, the processing consecutive in-phase sample signals or consecutive out-phase signals of the sample comprises application of the following equations:

$$Sin - {phase\_T2}^{*} corrected = Sin - phase1 \cdot \sqrt{Sin - phase1 / Sin - phase2} ; or$$

$$Sin - {phase\_T2}^{*} corrected = Sin - phase1 \cdot \sqrt{Sout - phase1 / Sout - phase2} ; or$$

$$Sout - {phase\_T2}^{*} corrected = Sout - phase1 \cdot \sqrt{Sin - phase1 / Sin - phase2} ; or$$

$$Sout - {phase\_T2}^{*} corrected = Sout - phase1 \cdot \sqrt{Sout - phase1 / Sout - phase2} .$$

In certain preferred embodiments, the present invention provides a method of imaging comprising providing a sample, and an MRI device. Any MRI device may be used or may be modified for use with the systems and methods of the present invention. In some embodiments the MRI device utilizes a clinical pulse sequence, wherein the clinical pulse sequence comprises a corrected T2* NMR relaxation effect value, wherein the corrected T2* NMR relaxation effect value is obtained through, for example, processing consecutive in-phase sample signals or consecutive out-phase signals of the sample. In other embodiments, a plurality of images of the sample is obtained with the MRI device. Any animal or tissue may be used as the sample. In some embodiments, the sample is a human head and neck, chest, abdomen, pelvis, or extremity. In other embodiments, the sample is an organ within the body, such as the liver. In other embodiments, the sample is normal tissue, abnormal tissue, or lesion within the body

In preferred embodiments, the images have reduced T2* NMR relaxation effect. In further embodiments, the processing of consecutive in-phase sample signals or consecutive out-phase signals of the sample comprises application of the following equations:

$$Sin - {phase\_T2}^{*} corrected = Sin - phase1 \cdot \sqrt{Sin - phase1 / Sin - phase2} . ; or$$

$$Sin - {phase\_T2}^{*} corrected = Sin - phase1 \cdot \sqrt{Sout - phase1 / Sout - phase2} ; or$$

$$Sout - {phase\_T2}^{*} corrected = Sout - phase1 \cdot \sqrt{Sin - phase1 / Sin - phase2} . ; or$$

$$Sout - {phase\_T2}^{*} corrected = Sout - phase1 \cdot \sqrt{Sout - phase1 / Sout - phase2} .$$

In other embodiments, the sample emits a signal, wherein the signal comprises proton species, wherein the proton species comprises water protons and fat protons. In other embodiments, the plurality of images comprise at least one pair of consecutive in-phase and out-phase images of the sample obtained with a low flip angle setting, and at least one pair of consecutive in-phase and out-phase images of the sample obtained with a high flip angle setting. In further embodiments, the low flip angle setting is approximately 20 degrees. In other embodiments, the high flip angle setting is approximately 70 degrees.

In even further embodiments, the apparent fat-percentage of the at least one pair of consecutive in-phase and out-phase images of the sample obtained with a low flip angle setting and at least one pair of consecutive in-phase and out-phase images of the sample obtained with a high flip angle setting is detected. In yet further embodiments, the dominant proton species within the sample is identified, wherein the water protons are dominant when the apparent fat-percentage increases as flip angle is increased for a sample image, and wherein the fat protons are dominant when the fat-percentage decreases as flip angle is increased for a sample image.

In other certain preferred embodiments, the present invention provides a method of imaging comprising the steps of providing a sample and an MRI device, wherein the sample emits a signal, wherein the signal comprises proton species, wherein the proton species comprises water protons and fat protons. In further embodiments, a plurality of images of the sample is obtained with the MRI device, wherein the plurality of images comprise at least one pair of consecutive in-phase and out-phase images of the sample obtained with a low flip angle setting, and at least one pair of consecutive in-phase and out-phase images of the sample obtained with a high flip angle setting.

In further embodiments, the apparent fat-percentage of the at least one pair of consecutive in-phase and out-phase images of the sample obtained with a low flip angle setting and at least one pair of consecutive in-phase and out-phase images of the sample obtained with a high flip angle setting is detected. In yet further embodiments, the dominant proton species within the sample is identified, wherein the water protons are dominant when the apparent fat-percentage increases as flip angle is increased for a sample image, and wherein the fat protons are dominant when the fat-percentage decreases as flip angle is increased for a sample image.

### DESCRIPTION OF THE FIGURES

FIG. 1 shows evolution of water and fat components of MRI signal as a function of echo time. During each τ the fat component evolves 180 degrees relative to the water component.

FIG. 2 shows application of Equation (4) to estimate % fat in simulation of low T1-weighting (20 degs) and high T1-weighting (70 degs) with and without T2* correction. Low T1-weighted estimates are relatively accurate if fat content is known to be below 50%. Values above 50% are mistakenly assigned to below 50% due to ambiguity of “magnitude format” data. Relaxation times were simulated to be T1water=600 ms; T1fat=300 ms; T2*water=20 msec; T2*fat=20 msec. SPGRE acquisition parameters simulated to be TR=150 msec; TE=2.3 msec (out-phase); and TE=4.6 msec (in-phase).

FIG. 3 shows performance of new algorithm to determine whether % fat is above or below 50% level by combination of two T1-weightings. Same conditions simulated as in FIG. 2. Algorithm accurately distinguishes above 50% from below 50% conditions aside from minor error near around 45-55%.

FIG. 4 presents a functional prototype of analysis software.

FIG. 5 presents a phantom experiment of continuously varying fat-percentage content by oblique slice through an oil/water interface. The dark band through “out phase” image is due to interference of water and fat components. Resultant apparent fat-percentage is shown in FIG. 6.

FIG. 6 presents oil/water phantom experiment results. Quantitative maps of apparent fat-percentage at low T1-weighting (left) and high T1-weighting (middle). Results of new algorithm to remove ambiguity about 50% level is represented in image on the right. Significant increase in dynamic range above 50% is achieved. The vertical line through the three images demonstrates general agreement with simulation is shown graphically below the images.

FIG. 7: Illustrates scan of the human abdomen acquired in two breath-hold periods (only 1 slice of 35 acquired slices through the liver is shown). The images on top are % fat calculated using low T1-weighting (e.g., Flip=20 degrees) and high T1-weighting (e.g., Flip=70 degrees).

### DEFINITIONS

To facilitate understanding of the invention, a number of terms are defined below.

As used herein, the term “magnetic resonance imaging (MRI) device” or “MRI” incorporates all devices capable of magnetic resonance imaging or equivalents. The methods of the invention can be practiced using any such device, or variation of a magnetic resonance imaging (MRI) device or equivalent, or in conjunction with any known MRI methodology. For example, in magnetic resonance methods and apparatuses, a static magnetic field is applied to a tissue or a body under investigation in order to define an equilibrium axis of magnetic alignment in a region of interest. A radio frequency field is then applied to that region in a direction orthogonal to the static magnetic field direction in order to excite magnetic resonance in the region. Magentic field gradients are applied to spatially encode the signals. The resulting signals are detected by radio-frequency coils placed adjacent to the tissue or area of the body of interest. See, e.g., U.S. Pat. Nos. 6,144,202; 6,128,522; 6,127,775; 6,119,032; 6,111,410; 5,555,251; 5,455,512; 5,450,010, each of which is herein incorporated by reference in its entirety. MRI and supporting devices are manufactured by, e.g., Bruker Medical GMBH; Caprius; Esoate Biomedica; Fonar; GE Medical Systems (GEMS); Hitachi Medical Systems America; Intermagnetics General Corporation; Lunar Corporation; MagneVu; Marconi Medicals; Philips Medical Systems; Shimadzu; Siemens; Toshiba America Medical Systems; and Varian; including imaging systems, by, e.g., Silicon Graphics.

As used herein, the term “sample” is used in its broadest sense. In one sense it can refer to a tissue sample. In another sense, it is meant to include a specimen or culture obtained from any source, as well as biological. In another sense, it is meant to include inanimate objects such as non-living items. In another sense, it is meant to include whole living systems (including humans).

As used herein, the term “biological entity” is used in its broadest sense. A biological entity may be obtained from animals (including humans) and encompass fluids, solids, organs, whole bodies, internal cavities, tissues, and gases. Biological samples include, but are not limited to whole organs, such as a brain, heart, lung, and the like; blood products, such as plasma, serum and the like; tissue products, such as skin, vulnerable plaque in carotid arteries, and the like. These examples are not to be construed as limiting the sample types applicable to the present invention.

As used herein, the terms “processor,” “imaging software,” “software package,” or other similar terms are used in their broadest sense. In one sense, the terms “processor,” “imaging software,” “software package,” or other similar terms refer to a device and/or system capable of obtaining, processing, and/or viewing images obtained with an imaging device.

### DETAILED DESCRIPTION

The present invention provides systems and methods for obtaining high resolution MR images. The present invention also relates to systems and methods for obtaining high resolution MR images with reduced NMR relaxation effects. The present invention also relates to systems and methods for obtaining high resolution MR images wherein fat-percentage ambiguity is reduced. In preferred embodiments, a high resolution MR image is obtained through obtaining the following data within a one-breath hold time period: (a) at least ten anatomical slices; (b) three to four echos that include at least one in-phase/out-phase pair, and one pair of consecutive in-phase or out-phase echos for T2* correction; (c) two flip angle settings for low and high T1-weighting to reduce apparent fat-percentage ambiguity and provide high quality anatomic images. Certain illustrative embodiments of the present invention are described below. The present invention is not limited to these specific embodiments.

The description is provided in the following sections: I) MR Imaging; II) MRI Device; III) MR Images With Reduced NMR Relaxation Effects; IV) MR Images With Reduced Fat-Percentage Ambiguity; V) MR Imaging of Disease; and VI) MRI Software.

I. MR Imaging

Magnetic resonance imaging (MRI) is extensively used for diagnostic imaging of a subject's anatomy, as well as functional assessment of tissues, organs and processes within a body or sample. Hydrogen protons in tissue water and hydrogen protons in tissue fat contribute to the MR signal represented in an MRI image. Hydrogen protons in tissue water and tissue fat have distinctive properties (e.g., NMR frequency differences) arising from respective chemical and physical environments. Collectively, these properties impact the relative intensities of tissue water and tissue fat in the final MR image.

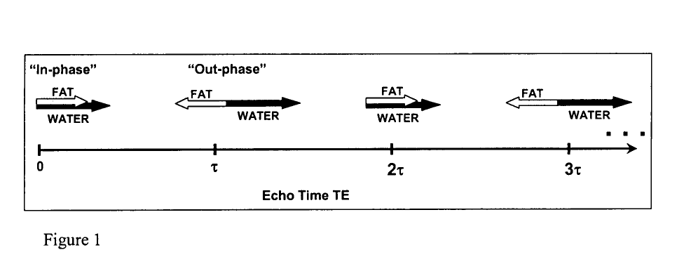

A property often used in MR imaging is the “chemical shift” between water and fat. Chemical shift determines the NMR frequency difference, Av, between water and fat for a given magnetic field strength. For example, the NMR frequency difference between water and fat is approximately 220 Hz on a 1.5 Tesla MRI system. This slight difference in NMR precessional frequency causes the relative orientation of fat and water magnetization vectors to change by 180 degrees over an interval τ=[1/(2Δν)]. The alternating co-directional (“in-phase”) and opposed direction (“out-phase”) states with each τ is presented in FIG. 1.

Chemical shift is used in conventional MRI to qualitatively assess fat involvement in a sample (e.g., body tissue). Typically, image sets are acquired such that water and fat constituents combine to yield a stronger signal (e.g., in-phase for increased image intensity) in tissue volume elements (e.g., voxels) where fat and water co-exist. Such images are visually compared to out-phase images where water and fat interfere to yield a weaker signal. By this model, voxels comprised of 100% water or 100% fat do not exhibit an intensity change between in-phase and out-phase images.

An approach that utilizes chemical shift in MR imaging isolates the water signal through addition of in-phase and out-phase images, and isolates the fat signal through subtraction of out-phase image from in-phase image (see, e.g., Dixon, Radiology, 153:189-194 (1984); herein incorporated by reference in its entirety). Unfortunately, there are important caveats to this model. Firstly, the vast majority of MR images are presented in “magnitude” format. That is, only the absolute values of the net in-phase and net out-phase signals are available. Thus, one cannot determine whether the net magnetization in an out-phase condition points along the fat or water directions. Consequently, addition and subtraction of in-phase (IP) and out-phase (OP) images is highly ambiguous to which species dominates a given voxel. Mathematically, this process is given by:
IP=|Fat+Water|; OP=|Fat−Water|
IP+OP=Water (when water is majority species)
IP+OP=Fat (when fat is majority species)
IP−OP=Water (when fat is majority species)
IP−OP=Fat (when water is majority species); Equation 1.

Nevertheless, the magnitude format method has been used to quantify fat and water fractions in phantoms, ground meat, and in vivo in the liver and adrenal glands, despite an intrinsic ambiguity regarding whether fat or water are the dominant species (see, e.g., Lee, et al., Radiology 1984; 153: 195-201; Heiken, et al., Radiology 1985; 157:707-710; Rosen, et al., Radiology 1985; 154:469-472; Buxton, et al., Magn. Reson. Med. 1986; 3:881-890; Mitchel, et al., Investigative Radiology 1991; 26:1041-1052; Levenson, et al., Am. J. Roentg. 1991; 156:307-312; Namimoto, et al., Radiology 2001; 218:642-646; Fishbein, et al., Magn. Reson. Imag. 1997; 15:287-293; Fishbein, et al., Magn. Reson. Imag. 1997; 15:287- 293; Fishbein, et al., Ped. Radiol. 2001; 31:806-809; each herein incorporated by reference in their entireties).

Processing MR images in magnitude format avoids imperfections in magnet homogeneity. Alternate methods exist which measure and correct for magnet inhomogeneities. Such methods permit in-phase and out-phase images to be calculated in a “phase-sensitive” format through the use of specialized algorithms. These specialized algorithms derive “pure fat” and “pure water” images using two or more image acquisitions that include combinations of in-phase, out-phase, and inhomogeneity estimation scans (see, e.g., Borrello, et al., Radiology 1987; 164:531-537; Szumowski, et al., Radiology 1994,192:555-561; Coombs, et al., Magn. Reson. Med. 1997; 38:884-889; each herein incorporated by reference in their entireties). These approaches generate separate anatomical images of water and fat, which are displayed in standard MRI format. That is, typically these “water-only” and “fat-only” images are visually interpreted side-by-side on film or a viewing workstation. While correctly labeled “water-only” and “fat-only,” these images are not quantitatively accurate maps of water/fat content in terms of percentages or in physical concentration units. In addition, image intensity values are arbitrarily scaled. There often is a strong spatial modulation of image intensity values due to spatial inhomogeneity of several hardware components. Tissue proximity to transmit and receive radiofrequency coils has a strong impact on image intensity.

An additional property often used in MRI imaging involves the NMR “relaxation” that differentially affects signals derived from water and fat. NMR relaxation hinders fat content quantification within existing MRI methods. The amount of fat and water signal produced depends on timing of the imaging sequence relative to tissue-inherent relaxation times (e.g., T1, T2, and T2*). NMR relaxation properties are influenced by tissue type (e.g., liver and kidney) and tissue state (e.g., normal and diseased). NMR relaxation properties embody the microscopic magnetic environment of the protons. Mathematically and experimentally, the role of NMR relaxation in MRI signal may be quantified. Yet, NMR relaxation times are rarely measured clinically because such measurement requires the acquisition of multiple image sets at various acquisition time combinations for mathematical reduction. This can lead to unacceptably long clinical exam times and/or complications due to tissue/organ motion during the measurement interval. In lieu of this, a subset of conditions may be acquired to accentuate, or “weight”, NMR relaxation influences. It is the heavily “T1-weighted”, “T2-weighted”, and “T2*-weighted” image sets that are preferred for diagnostic interpretation because such image sets have the greatest contrast and clarity. In terms of accurate fractional fat quantification, a “proton-density-weighted” image is desired. Proton-density-weighted MRI pulse sequences exist and are available for routine MRI examination.

II. MRI Device

The present invention provides systems and methods of MRI scanning. The present invention is not limited to a particular MR imaging device. In preferred embodiments, the present invention provides an MRI device with an ability to scan large body regions at high spatial resolution. In further preferred embodiments, the body region is the chest, abdomen, pelvis, or extremity of a subject. In further preferred embodiments, the body region is an organ within the body, such as the liver of a subject. In other embodiments, the sample is normal tissue, abnormal tissue, or lesion within the body.

The MRI device is able to image a sample in a short amount of time. In preferred embodiments, the MRI device is configured to scan a sample in two-breath hold time periods. In further preferred embodiments, the MRI imaging device is configured to scan a sample in a one breath-hold time period.

The MRI device provides a clinical pulse sequence for scanning samples. The present invention is not limited to a particular clinical pulse sequence for scanning samples. In some embodiments of the present invention, the MRI device utilizes the 2D spoiled gradient-recalled-echo (hereinafter “SPGRE”) clinical pulse sequence for scanning a sample. A signal derived from a given voxel of tissue via the SPGRE sequence is given by:
S(TR,TE, θ)=H·P[(1−exp(−TR/T1)]·sin(θ)·exp (−TE/T2*))/[1−exp(−TR/T1)·cos(θ)]; Equation 2;
where TR and TE are machine settings related to timing of the pulse sequence; θ is the excitation flip angle used to invoke the NMR signal; T1 and T2* are the tissue-specific relaxation times; H includes all the hardware and systematic influences on the signal; and P is the desired proton density. Echo time is controlled to impart in-phase and out-phase conditions between water and fat. That is, TE=nτ; where n is an even integer for in-phase states and odd for out-phase states. Recall, τ is determined by the chemical shift difference between water and fat that is known for a given MRI field strength (e.g., τ=2.27 msec on a 1.5 Tesla magnet).

The MRI device generates sample images. In some embodiments, the MRI device generates MR images with reduced magnet inhomogeneity effects. In preferred embodiments, the MRI imaging device generates magnitude format images as this substantially simplifies processing and removes magnet inhomogeneity effects.

The MRI device processes in-phase signals and out-phase signals in generating an MR image. The present invention is not limited to a particular method of obtaining in-phase and out-phase signals. In preferred embodiments, in-phase and out-phase signals inclusive of relaxation are given by:
Sᵢₙ₋ₚₕₐₛₑ=|H·(P_{water}·R_{water}+P_{fat}·R_{fat})|Sₒᵤₜ₋ₚₕₐₛₑ=|H·(P_{water}·R_{water}−P_{fat}·R_{fat})|; Equation 3;
where P_{fat} and P_{water} are the targeted densities of fat and water, and R_{water} and R_{fat} include all NMR relaxation effects.

The MRI device generates images in which fractional fat and fraction water compositions within a sample are estimated. The present invention is not limited to particular methods of estimating fractional fat and fractional water compositions within a sample. In some preferred embodiments, the fractional fat and water compositions within a sample are estimated by the following equations:
[(Sᵢₙ₋ₚₕₐₛₑ+Sₒᵤₜ₋ₚₕₐₛₑ)·100%]/[2·Sᵢₙ₋ₚₕₐₛₑ]=% water when water is majority species
[(Sᵢₙ₋ₚₕₐₛₑ+Sₒᵤₜ₋ₚₕₐₛₑ)·100%]/[2·Sᵢₙ₋ₚₕₐₛₑ]=% fat when fat is majority species
[(Sᵢₙ₋ₚₕₐₛₑ−Sₒᵤₜ₋ₚₕₐₛₑ)·100%]/[2·Sᵢₙ₋ₚₕₐₛₑ]=% water when fat is majority species
[(Sᵢₙ₋ₚₕₐₛₑ−Sₒᵤₜ₋ₚₕₐₛₑ)·100%]/[2·Sᵢₙ₋ₚₕₐₛₑ]=% fat when water is majority species; Equation 4.

III. MR Images With Reduced NMR Relaxation Effects

The present invention is designed to generate images with reduced T1 NMR relaxation effects. The present invention is not limited to particular scanning parameter(s) for reducing T1 NMR relaxation effects. In some embodiments, undesired hardware effects are reduced through normalization of H with Sᵢₙ₋ₚₕₐₛₑ. In other embodiments, T1 NMR relaxation effects are reduced through increasing TR and/or reducing θ. In some embodiments, a scan image matrix resolution of 128-256 is obtained. In preferred embodiments, scans obtained with a 15-30 second scan time length and a TR value of 100-200 msec result in image matrix resolution of 128-256. In other preferred embodiments, scans obtained with a flip-angle reduced to 20° or less and a TR value of 100-200 msec result in reduced T1 NMR relaxation effects.

The present invention is designed to generate images with reduced T2* NMR relaxation effects. The present invention is not limited to particular scanning parameter(s) for reducing T2* NMR relaxation effects. In preferred embodiments, T2* NMR relaxation effects are reduced through use of an “effective T2*” value applicable to both water and fat signals. There is justification in using a single effective T2* considering that magnetic inhomogeneity on the scale of a voxel is a strong contributor to T2* that impacts both water and fat T2*. Estimation of the effective T2* for each voxel is accomplished through collection of at least two in-phase or two out-phase gradient echos. The ratio of signals from a pair of consecutive in-phase echos (Sᵢₙ₋ₚₕₐₛₑ₁/Sᵢₙ₋ₚₕₐₛₑ₂) or pair of out-phase echos (Sₒᵤₜ₋ₚₕₐₛₑ₁/Sₒᵤₜ₋ₚₕₐₛₑ₂) reflect T2* signal loss over a 2 τ interval. In preferred embodiments, the correction for T2* signal loss between out-phase and in-phase echos is applied to the in-phase data as follows:

$$Sin - {phase\_T2}^{*} corrected = Sin - phase1 \cdot \sqrt{Sin - phase1 / Sin - phase2} ; or \\ Sin - {phase\_T2}^{*} corrected = Sin - phase1 \cdot \sqrt{Sout - phase1 / Sout - phase2} . \qquad Equation 5$$

Alternatively, the signal loss between in-phase and out-phase echos is applied to the out-phase data as follows:

$$Sout - {phase\_T2}^{*} corrected = Sout - phase1 \cdot \sqrt{Sin - phase1 / Sin - phase2} ; or \\ Sout - {phase\_T2}^{*} corrected = Sout - phase1 \cdot \sqrt{Sout - phase1 / Sout - phase2} . \qquad Equation 6.$$

Equation 5 is used to correct the first in-phase echo when it is recorded at a later echo time relative to the first out-phase echo. Alternatively, Equation 6 is used to correct the first out-phase echo when it is recorded at a later echo time relative to the first in-phase echo. In preferred embodiments, at least two in-phase and/or two out-phase echos are collected within TE=10 msec. In such embodiments, the quantity of slices acquired in a single pass is TR/TE=(100-200)msec/10 msec=(10-20) slices (yet more slices are possible for TE<10 msec). In such embodiments, this quantity is sufficient to completely scan a large sample (e.g., a human liver) at acceptable spatial resolution in one or two breath-hold periods.
IV. MR Images With Reduced Fat-Percentage Ambiguity

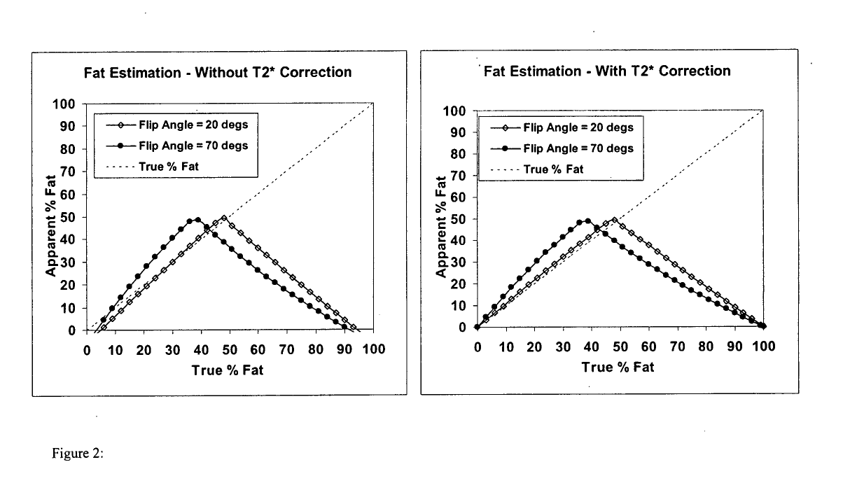

The present invention is designed to provide MR images with reduced fat-percentage ambiguity and to provide images showing fat-percentage in a single image. In some embodiments, the scanning parameters of the present invention are designed to reduce fat-percentage ambiguity. The present invention is not limited to particular scanning parameter(s) for reducing fat-percentage ambiguity. Generally, fat exhibits shorter native T1 than water in soft tissues. In addition, as displayed in FIG. 2, apparent fat-percentage increases with increasing T1 weighting (e.g., from lower to higher flip angle θ ) when the fat is the minority component (0 to approximately 40%). If, however, the apparent fat-percentage decreases with increased T1-weighting then fat is determined to be the majority component (i.e. over 50%). Comparison of images scanned at low and high T1 weighting reduces the fat-percentage ambiguity without complex phase-sensitive processing and obviates the need to formally calculate tissue T1's. In preferred embodiments, a sample is scanned with a low T1-weighting (e.g., θ approximately 20 degrees in a SPGRE sequence with TR=100-200 msec) and a high T1-weighting (θ approximately 70 degrees in a SPGRE sequence with TR=100-200 msec). In further preferred embodiments, detection of fat as the major or minor constituent in a sample image is determined through comparing the fat-percentage at low and high T1-weighting.

In preferred embodiments, the following data is acquired within a one breath-hold period: (a) at least ten anatomical slices; (b) three to four echos that include at least one in-phase/out-phase pair, and one pair of consecutive in-phase or out-phase echos for T2* correction; (c) two flip angle settings for low and high T1-weighting to reduce apparent fat-percentage ambiguity and provide high quality anatomic images. For example, in the abdomen it is desirable to have the data from a given sample region acquired within one breath-hold so that images are inherently spatially registered prior to any mathematical combination. If more than one breath-hold is required to cover the target anatomy, adjacent anatomical zones are scanned in separate breath-holds. For example, 15 slices are acquired in 38 seconds using a TR=150 msec and TEs10 msec (38 sec=150 msec×128×2 flip angles). Scan time is reduced and/or resolution increased through incorporation of several compatible techniques. Compatible techniques include: (a) “rectangular field-of-view” reduces scan time by 0.5-0.8 factor; (b) “partial-Fourier” reduces scan time by 0.6-0.75 factor; and (c) parallel imaging reduces scan time by 0.25-0.5 factor. In cases where breath-hold intervals are too long and the subject is unable to hold their breath, additional respiratory monitored/triggered techniques are applicable. In addition, this method is easily applicable to other anatomic sites such as the head and neck, brain, spine, pelvis, extremities, and breast where motion is less an issue. In these applications, image quality can be improved by signal averaging and increased resolution without the breath-hold limitations.

V. MR Imaging of Disease

The present invention is useful in imaging and diagnosing diseases within a body region. The present invention is not limited to a particular disease. In preferred embodiments, the present invention is useful in imaging diseases characterized by fatty infiltration of a body region, organ (e.g., liver), or tissue. In further embodiments, the present invention is useful in imaging nonalcoholic fatty liver disease (NAFLD). In some preferred embodiments, the present invention is useful in imaging adrenal masses where fat involvment suggest a benign lesion. In other preferred embodiments, the present invention is useful in imaging breast density. In particular, by providing single images showing fat-percentage and summary statistics of fat involvement, medical practitioners can more easily diagnose conditions, as well as risk of future disease. In particular, by comparing changes in such images, for example, over time and/or in response to medical interventions (e.g., treatment with drugs), useful information is readily obtained that would be difficult or impossible to access by water-only and fat-only images. Progression of disease is also readily observed.

As noted, the present invention is particularly applicable in the imaging of NAFLD. NAFLD is the most common liver disease in the United States with a prevalence of approximately 5% in the general population and up to 25% to 75% in patients with obesity and type II diabetes mellitus. NAFLD refers to a wide spectrum of liver damage, ranging from simple steatosis to steatohepatitis (NASH), advanced fibrosis, and cirrhosis. Obesity, type 2 (non-insulin-dependent) diabetes mellitus, and hyperlipidemia are coexisting conditions frequently associated with nonalcoholic fatty liver disease. Given its high incidence in the general population, NAFLD is now considered the most common cause of cryptogenic cirrhosis.

NAFLD is characterized histologically by steatosis, mixed inflammatory-cell infiltration, hepatocyte ballooning and necrosis, glycogen nuclei, Mallory's hyaline, and fibrosis. The presence of these features, alone or in combination, accounts for the wide spectrum of nonalcoholic fatty liver disease.

A net retention of lipids within hepatocytes, mostly in the form of triglycerides, is a prerequisite for the development of NAFLD. The primary metabolic abnormalities leading to lipid accumulation are not well understood, but could consist of alterations in the pathways of uptake, synthesis, degradation, or secretion in hepatic lipid metabolism resulting from insulin resistance. Increased intrahepatic levels of fatty acids provide a source of oxidative stress, which may be responsible for the progression from steatosis to steatohepatitis to cirrhosis. Thus, although symptoms of liver disease rarely develop in patients with fatty liver who are obese, have diabetes, or have hyperlipidemia, the steatotic liver may be vulnerable to further injury when challenged by additional insults. Progression from simple steatosis to steatohepatitis and to advanced fibrosis results from two distinct events. First, insulin resistance leads to the accumulation of fat within hepatocytes, and second, mitochondrial reactive oxygen species cause lipid peroxidation, cytokine induction, and the induction of Fas ligand.

There is also growing evidence that NAFLD contributes to the progression of other liver diseases. Hepatic steatosis related to visceral adiposity is a major independent risk factor for fibrogenesis related to chronic hepatitis C infection, whereas viral burden has no relevance to disease progression. The diagnosis of NAFLD is suspected in persons with asymptomatic elevation of aminotransferase levels, radiologic findings of fatty liver, or unexplained persistent hepatomegaly. The clinical diagnosis and liver tests have a poor predictive value with respect to histologic involvement. Current imaging studies do not provide an accurate quantification of the amount of fat in the liver or determine the severity of liver damage. A clinical suspicion of nonalcoholic fatty liver disease and its severity is only be confirmed with a liver biopsy.

A difficult management decision in clinical hepatology, involves whether to perform a liver biopsy in a patient with abnormal liver function tests, particularly in the absence of diagnostic serology, or a history of drug or alcohol use. The benefits of performing an invasive procedure in such asymptomatic patients should be balanced against the potential hazards of bile leak or hemorrhage, and patient discomfort.

Proposed treatments for NAFLD include modification of the clinical conditions associated with NASH including type II diabetes mellitus, hyperlipidemia, and obesity. Weight reduction can improve liver enzyme abnormalities, and may improve liver histology in patients with NASH. Regression of hepatic steatosis and the associated inflammatory process are features used to assess response to therapy. Currently, repeat biopsy is the only reliable means to assess such changes.

The severity of fatty infiltration has been proposed as a risk factor for the progression of simple steatosis to NASH. Currently, no simple and reliable imaging method is available to quantitatively determine the degree of fatty infiltration, necessitating tissue biopsy for the initial diagnosis, and the assessment of response to treatment. Liver biopsy is an invasive procedure that is associated with significant morbidity. Moreover, biopsy only samples a relatively minute amount of tissue. If the tissue/organ of interest is not homogeneous, the biopsy sample may miss the actual region of disease or representative tissue. The present invention not only provides a non-invasive alternative to biopsy, it also provides the means to select the appropriate site for biopsy in the event further histological characterization is needed.

The present invention provides a non-invasive option for the assessment of NAFLD. By applying the MR imaging systems and methods of the present invention for quantitative assessment of fatty infiltration of the liver, the number of biopsies required to monitor changes in the liver in response to treatment is reduced.

VI. MRI Software

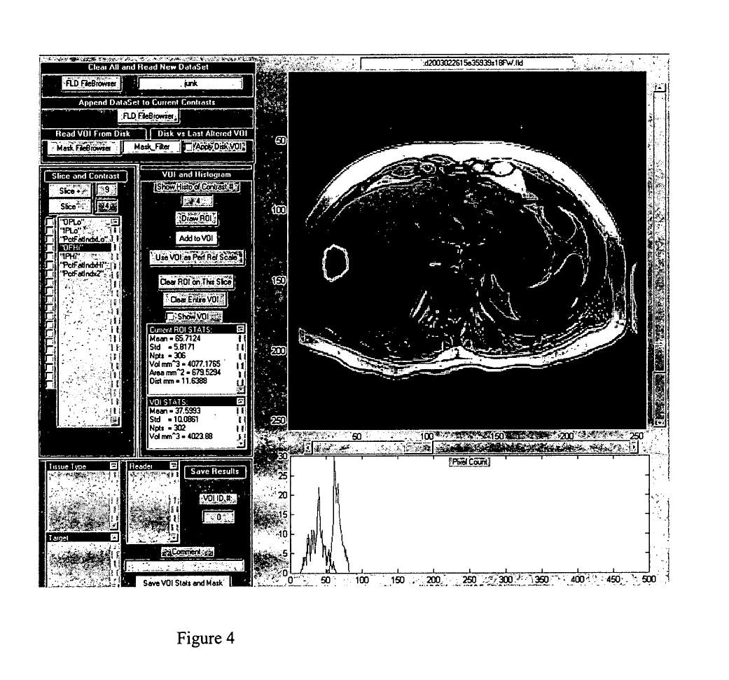

A software package is further provided for obtaining, processing, and viewing images obtained with the present invention. The present invention is not limited to particular type of software package. In preferred embodiments, the software package provides: (a) access to a clinical MR image database; (b) reconstruction of apparent fat-percentage images for each slice through the scanned anatomy using the algorithm steps described above; (c) quantitative display and hardcopy of single- or multi-slice apparent fat-percentage images via color or other display formats; (d) quantitative analysis of user-defined regions and volumes of interest using the aid of vectorized 3D images; and (e) automatic or semi-automatic quantitative analysis of regions and volumes to generate summaries statistics of fat involvement (e.g., fractional amount of the whole tissue/organ that has a fat-percentage above specified threshold). The term “vector” refers, for example, to a format where all points of an imaged object are represented by an array of available contrasts. Such contrasts include, but are not limited to, the original (e.g., T1-weighted) and the derived images (e.g., apparent fat-percentage). In some embodiments, the software package is designed to permit a user to interact with the vector image space through a single viewport. In further embodiments, interaction with the vector image space through a single viewport permits a user is to (a) delineate specific regions of interest based on an ensemble of contrasts; and (b) confirm the specific region of interest location through overlay and application with additional contrasts. In even further embodiments, volumes of interest are generated from regions of interest across multiple slices thereby facilitating analysis of three-dimensional structures (see, e.g., FIG. 4). In some embodiments, the software is configured to compare two or more images showing fat-percentage content to highlight relevant changes/differences between the images (e.g., to highlight regions of tissues or subjects that show change).

### EXAMPLES

### Example 1

FIG. 2 illustrates apparent fat-percentage, as given by Equation 4, as a function of true % fat at low T1-weighting (θ=20 degrees) and high T1-weighting (θ=70 degrees). As expected, the lower flip angle data (faint line) more faithfully matches the true % fat (dashed line) due to reduced T1-relaxation contamination. Also note the persistent ambiguity in % fat that originates from the magnitude format of in-phase and out-phase data. For example, both 30% and 70% true fat content yield apparent fat content around 30%.

### Example 2

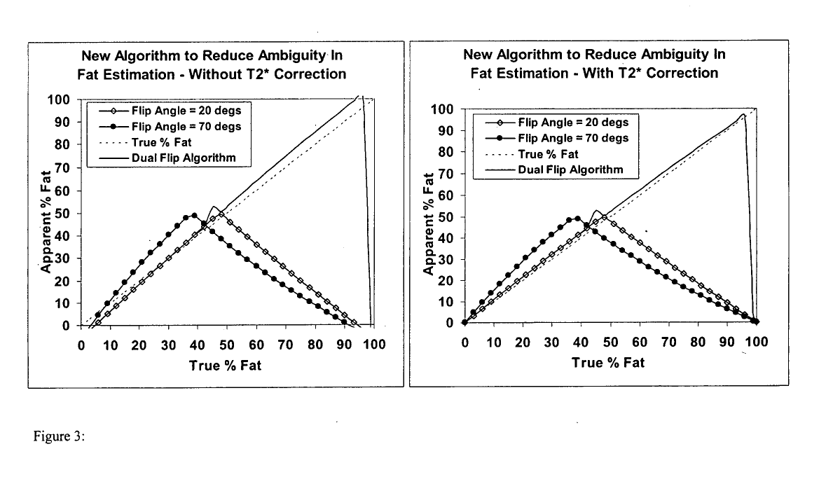

FIG. 3 illustrates algorithm performance using the conditions simulated in FIG. 2. Note the algorithm substantially improved the dynamic range of discernible apparent fat-percentage values. The algorithm incorrectly classified mixtures 45 to 49% as being −55 to 51% respectively. In most applications this error is not considered clinically significant given that current fat indices are coarse subjective scales, such as provided by pathology reading of biopsies by terms “mild- moderate-severe.” The algorithm also has difficulty distinguishing very low (e.g., 0 to 5%) from very high (e.g., 95 to 100%) mixtures of fat.

### Example 3

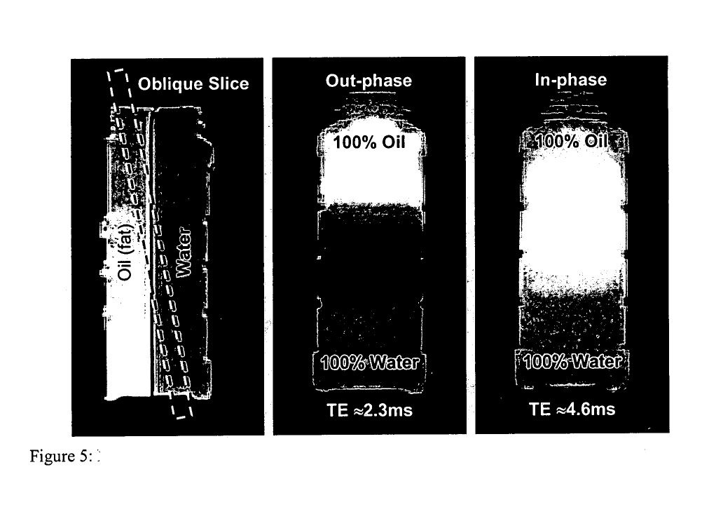

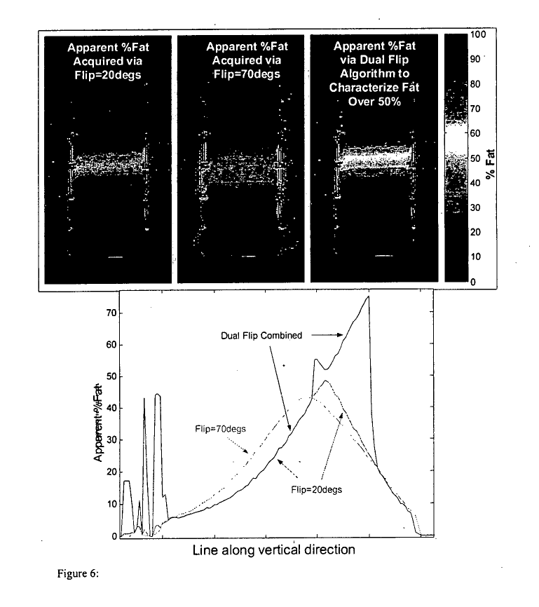

A water/fat phantom was scanned and data processed within the scanning parameters described in the present invention in order to illustrate the basic elements of the invention. The phantom consisted of a bottle (approximate size 700 ml) filled with equal volumes of water and mineral oil (to simulate fat). A single oblique slice was prescribed to intersect the oil-water interface such that there was a continuous transition from pure water to pure oil as illustrated in FIG. 5. The signal cancellation effect in voxels that have comparable mix of fat and water is clearly apparent on the out-phase image. These data were acquired at two T1-weightings (20° and 70° flip angle) for generation of apparent % fat maps by the algorithms described which are shown as apparent % fat maps in FIG. 6. While not yet optimized, it is clear the algorithm successfully adds significant dynamic range above the 50% apparent fat level.

### Example 4

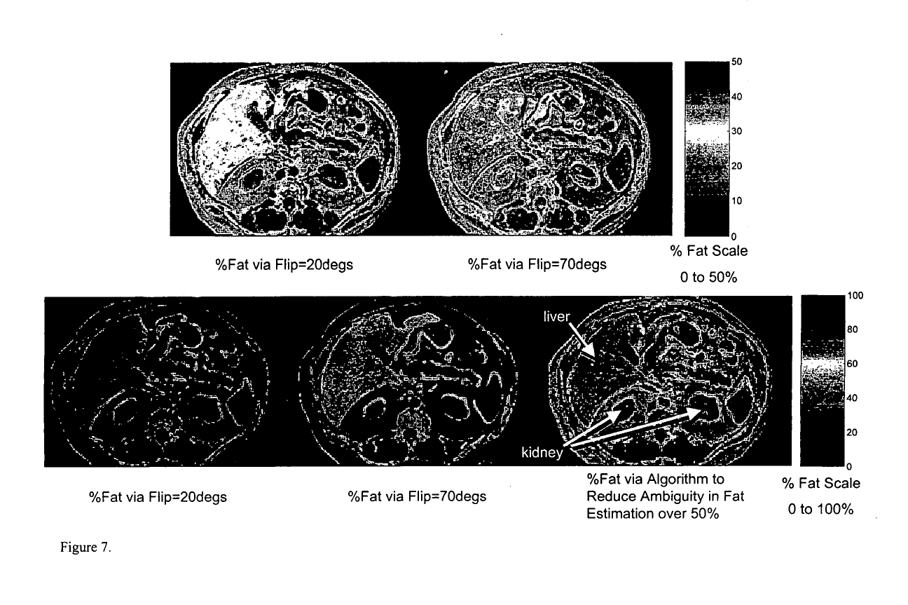

The abdomen of a human subject was scanned to acquire in-phase and out-phase conditions at two T1-weightings in two breath-hold periods (e.g., 1 slice of 35 acquired slices through the liver is shown in FIG. 7). The images on top of FIG. 7 are % fat calculated using low T1-weighting (e.g., Flip=20 degrees) and high T1-weighting (e.g., Flip=70 degrees). The percentage fat scale is 0 to 50% (e.g., the images do not characterize fat above 50%). Application of algorithms of the present invention (e.g., Equations 5 and 6) reduce % fat ambiguity by combination of two T1-weightings (e.g., the image on the lower right in FIG. 7 is calculated). The bottom panel in FIG. 7 illustrates the new image, along with original % fat via low and high T1-weighting, shown on a 0 to 100% fat scale. The apparent increase in % fat with increased T1-weighting confirms the fat content of the liver is below 50%. Water-dominant tissues (e.g., liver, kidneys, and muscle in this subject) and fat-dominant tissues (e.g., fat surrounding the kidneys) are now properly represented. A quantitative color scale allows rapid visual assessment of the level and distribution of fat content. Manual, semi-automatic, and automatic quantitative region-of-interest analysis may also be performed (e.g., ROI analysis indicates average liver fat is 31%).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in relevant fields, are intended to be within the scope of the following claims.

1. A system, comprising:
a) an MRI device, and
b) software, wherein said software is configured to receive data obtained from said MRI device, wherein said data comprise at least one pair of consecutive in-phase and out-phase echos of a sample, wherein said software is further configured to process said at least one pair of consecutive in-phase and out-phase echos, wherein said processing comprises generating a percent of fat content within a sample, wherein said software is further configured to display said fat percentage within said sample.

2. The system of claim 1, wherein said sample is selected from the group consisting of a human head and neck, a human chest, a human abdomen, a human pelvis, and a human extremity.

3. The system of claim 1, wherein said sample is a human liver.

4. The system of claim 1, wherein said sample is abnormal tissue or lesion.

5. The system of claim 1, wherein said data obtained from said MRI device comprises:
a) at least one image obtained with a low flip angle; and
b) at least one image obtained with a high flip angle.

6. The system of claim 5, wherein said low flip angle setting is 20 degrees.

7. The system of claim 5, wherein said high flip angle setting is 70 degrees.

8. The system of claim 1, wherein said MRI device is configured to analyze a clinical pulse sequence, wherein said clinical pulse sequence comprises a corrected T2* NMR relaxation effect value, wherein said corrected T2* NMR relaxation effect value is obtained through processing consecutive in-phase sample echos or consecutive out-phase echos of said sample.

9. The system of claim 8, wherein said processing consecutive in-phase sample signals or consecutive out-phase signals of said sample comprises application of an equation selected from the group consisting of:
$$Sin - {phase\_T2}^{*} corrected = Sin - phase1 \cdot \sqrt{Sin - phase1 / Sin - phase2} ; and$$
$$Sin - {phase\_T2}^{*} corrected = Sin - phase1 \cdot \sqrt{Sout - phase1 / Sout - phase2} ; and$$
$$Sout - {phase\_T2}^{*} corrected = Sout - phase1 \cdot \sqrt{Sin - phase1 / Sin - phase2} ; and$$
$$Sout - {phase\_T2}^{*} corrected = Sout - phase1 \cdot \sqrt{Sout - phase1 / Sout - phase2} .$$

10. A system, comprising software, wherein said software is configured to receive data obtained from a MRI imaging device, wherein said data comprise at least one pair of consecutive in-phase or out-phase echos of a sample, wherein said software is further configured to process said at least one pair of consecutive in-phase or out-phase echos, wherein said processing comprises generating a percent of fat content within a sample, wherein said software is further configured to display said fat percentage within said sample.

11. The system of claim 10, wherein said sample is selected from the group consisting of a human head and neck, human chest, a human abdomen, a human pelvis, and a human extremity.

12. The system of claim 10, wherein said sample is a human liver.

13. The system of claim 10, wherein said sample is abnormal tissue or lesion.

14. The system of claim 10, wherein said data obtained from said MRI device comprises:
a) at least one image obtained with a low flip angle; and
b) at least one image obtained with a high flip angle.

15. The system of claim 10, wherein said low flip angle setting is 20 degrees.

16. The system of claim 10, wherein said high flip angle setting is 70 degrees.

17. The system of claim 10, wherein said MRI imaging device is configured to analyze a clinical pulse sequence, wherein said clinical pulse sequence comprises a corrected T2* NMR relaxation effect value, wherein said corrected T2* NMR relaxation effect value is obtained through processing consecutive in-phase sample echos and consecutive out-phase echos of said sample.

18. The system of claim 15, wherein said processing consecutive in-phase sample signals and consecutive out-phase signals of said sample comprises application of an equation selected from the group consisting of:
$$Sin - {phase\_T2}^{*} corrected = Sin - phase1 \cdot \sqrt{Sin - phase1 / Sin - phase2} ; and$$
$$Sin - {phase\_T2}^{*} corrected = Sin - phase1 \cdot \sqrt{Sout - phase1 / Sout - phase2} ; and$$
$$Sout - {phase\_T2}^{*} corrected = Sout - phase1 \cdot \sqrt{Sin - phase1 / Sin - phase2} ; and$$
$$Sout - {phase\_T2}^{*} corrected = Sout - phase1 \cdot \sqrt{Sout - phase1 / Sout - phase2}$$

19. A method of generating a percentage of fat within a sample, comprising using the system of claim 1.

20. A method of generating a percentage of fat within a sample, comprising using the system of claim 10.
